(19) United States
(12) Patent Application Publication
(10) Pub. No.: US 2021/0153796 A1
(43) Pub. Date: May 27, 2021

(54) CONTROL DEVICE, SYSTEM, AND METHOD FOR DETERMINING A COMFORT LEVEL OF A DRIVER

(71) Applicants: TOYOTA MOTOR EUROPE, Brusseis (BE); INSTITUT FRANCAIS DES SCIENCES ET TECHNOLOGIES DES TRANSPORTS, DE L'AMENAGEMENT ET DES RESEAUX, Champs-sur-Marne (FR)

(72) Inventors: Marleen DE WESER, Brussels (BE); Hugo LOECHES DE LA FUENTE, Marseille (FR); Catherine BERTHELON, Salon-de--Provence (FR); Christophe JALLAIS, Lyon (FR); Alexandra FORT, Chuzelles (FR); Virginie ETIENNE, Lyon (FR)

(73) Assignee: TOYOTA MOTOR EUROPE, Brussels (BE)

(21) Appl. No.: 16/613,489

(22) PCT Filed: May 15, 2017
(86) PCT No.: PCT/IB2017/000801

§ 371 Date: Nov. 14, 2019

(51) Int. Cl. A61B 5/18; A61B 5/318; A61B 5/0205; A61B 5/0531; B60W 40/08

(52) U.S. Cl. CPC A61B 5/18; A61B 5/318; A61B 5/02405; A61B 5/0531; B60W 40/08; A61B 5/0205

(57) ABSTRACT

A control device, system, and method for a vehicle to determine a driver's comfort level, configured to receive a first sensor output of a first physiological sensor measuring at least one first physiological feature of the driver, and receive a second sensor output of a second physiological sensor measuring at least one second physiological feature of the driver, create at least one reference data set by recording the first sensor output over a first predetermined reference time period and recording the second sensor output over a second predetermined reference time period being different than the first predetermined reference time period, determine at least one reference index for the comfort level of the driver based on the reference data set, and determine a comfort level index value of the driver, the index value determined as a function of the reference index and the current first and/or second sensor output.

### FIELD OF THE DISCLOSURE

The present disclosure is related to a control device, system, and method for a vehicle for determining a comfort level of a driver, in particular, for measuring and quantifying online the experience of (dis)comfort during driving.

### BACKGROUND OF THE DISCLOSURE

Currently, new technologies in the automotive industry increasingly integrate numerous and complex safety systems. These safety systems can be divided into two main categories: passive systems designed to minimize the severity of an accident (such as seat belts, airbags, etc.), and active systems designed to minimize the risk of occurrence of an accident. The advanced driver assistance systems (ADAS) fall into this latter category. ADAS are designed to improve driving safety by reducing the risk of human error. These embedded systems can interact with the driver in different ways, either by giving the driver additional information on the state of the environment (via a multi-modal communication interface: audio, visual, tactile, etc.), or by processing information about the driver's mental state such as stress, fatigue, vigilance, or drowsiness in order to assist him and/or to prevent potential risks (cf. Jinjun Wang et al, 2010: “Real-time driving danger-level prediction”, Engineering Applications of Artificial Intelligence; Volume 23, Issue 8, December 2010, Pages 1247-1254). These existing systems process individually stress, fatigue, vigilance, or drowsiness by considering behavioural or physiological parameters separately.

However, it is desirable to not only measure one specific mental state of the driver (fatigue or drowsiness, etc.) but to measure the feeling or experience of discomfort while driving. Meng and Siren (2012) propose that discomfort while driving can be considered as a form of awareness of the driver and influences his/her own driving ability (cf. Meng and Siren (2012): “Cognitive problems, self-rated changes in driving skills, driving-related discomfort and self-regulation of driving in old drivers” Accident Analysis & Prevention, Volume 49, November 2012, Pages 322-329). Thus, these authors define the feeling of discomfort as related to a feeling of anxiety as a consequence of being self-aware that one's driving ability in a specific driving situation (for example driving in heavy traffic) may include a potential driving risk. This feeling of discomfort may increase in time and can lead to complete avoidance of specific driving situations. However, Meng and Siren only analyze subjective data using questionnaires without considering measuring the (objective) driver behavioural or physiological data. In addition, most of the research dealing with (dis)comfort while driving, addresses this notion mainly in terms of physical ergonomics of the driving cabin (posture, driver seat dimensions, etc.).

It is known to measure the driver's mental state by electrophysiological and/or driver behavioral measures. For example, WO2008127465 (A1) discloses a system which predicts driving danger by capturing vehicle dynamic parameter, driver physiological data and driver behaviour feature; applying a learning algorithm to the features; and predicting driving danger.

Another example is German Patent Application Publication number DE 10 2015 105494 A1 which discloses applying physiological information by receiving physiological data from the user of the vehicle, data from the vehicle, data regarding the driving situation, and then processing that information to determine the state of the user.

European Patent Application Publication number EP 2 591 969 A1 discloses a method for operating a vehicle system of a motor vehicle for determining at least one state variable describing the state of a driver, in particular his attention and/or his fatigue, and at least one describing a reference state, in particular a normal state, of the driver Reference data set.

Further, German Patent Application Publication number DE 10 2008 042342 A1 discloses a method for judging the attention of a driver of a vehicle, a control device using this method, and finally a corresponding computer program product for evaluating the attentiveness of the driver.

### SUMMARY OF THE DISCLOSURE

Currently, it remains desirable to provide a control device and method for determining a comfort level of a driver, in particular, to detect if the driver is not coping with driving conditions.

The invention applies a concept of (dis)comfort in driving which includes the multiple dimensions of (dis)comfort. These dimensions of (dis)comfort involve together body and mind and can be associated with mental wellness. Discomfort is thus defined as ‘feeling uncomfortable, uneasy, tense when one's wellbeing is disturbed by a given situation’, and can be measured/quantified when considering the driver's physiological and behavioral data.

Therefore, according to the embodiments of the present disclosure, a control device for a vehicle for determining a comfort level of a driver is provided. The control device being configured to:
receive a first sensor output of a first physiological sensor measuring at least one first physiological feature of the driver, and
receive a second sensor output of a second physiological sensor measuring at least one second physiological feature of the driver,
create at least one reference data set by recording the first sensor output over a first predetermined reference time period and recording the second sensor output over a second predetermined reference time period different than the first predetermined reference time period,
determine at least one reference index for the comfort level of the driver based on the at least one reference data set, and
determine a comfort level index value of the driver, the index value being determined as a function of the at least one reference index and the current first and/or second sensor output.

By providing such a control device, it is possible to measure and quantify online the experience of (dis)comfort during driving. Accordingly, the (dis)comfort of the driver can be determined, in order to anticipate a negative long-term impact on the driver's behaviour.

Importantly, discomfort can be detected during driving situations and that the discomfort can persist after these driving situations. Discomfort can also be understood as a dynamical modification of the driver's mental state (i.e., when the mental state is affected) that is produced during a given driving situation and that could persist after it. It includes that the feeling of discomfort can evolve and grow into a stronger negative condition during the period following the driving situation. As such, the feeling of discomfort can become a ‘residual general condition’. With the control device according to the disclosure it is possible to early detect discomfort in real-time, in order to avoid a stronger negative condition which may persist and even increase afterwards.

Moreover, by providing such a control device, the driving context can be taken into account to assess (dis)comfort in driving. Accordingly, it is possible to compare the current sensor outputs relating to the current specific driving situation with a specific reference index which is based on reference data relating to the same specific driving situation. Consequently, the (dis)comfort can be determined more precisely.

The inventors have determined that discomfort can be classified into two categories of discomfort: short term discomfort (shorter than 30 seconds) which includes unexpected driving events (pedestrian walking into the street); and long term discomfort (around 10 minutes) which includes discomfort by cumulative effect after series of uncomfortable situations. A complementarity between physiological features can be shown which allows the measurement of these types of discomfort:
Amplitude of normalized skin conductance level (SCL) is more appropriate to detect (dis)comfort during short terms and unexpected driving events.
Heart rate variability is more appropriate to detect discomfort within a longer time period or to assess the cumulative effect of a series of uncomfortable situations.

The control device may be configured to receive driving context information from a driving context detection unit, determine at least one reference index for the comfort level of the driver based on the at least one reference data set and, based on the current driving context information, determine the comfort level index value as a function of a first reference index and the current first sensor output or a second reference index and the current second sensor output.

In particular, the control device may be configured to determine a driving context event based on the driving context information. Such a driving context event may be for example a pedestrian crossing the road, a left turn of the vehicle and/or overtaking another vehicle.

Accordingly, the index value may be determined as a function of the driving context event, and current sensor outputs.

The driving context detection unit may use a combination of sensors, radar, GPS, and cameras, in order to generate the driving context information. The driving context detection unit may further be configured to interpret/recognize the current driving situation and generate the driving context information based on the result.

By providing such a control device, physiological information contained in the sensor outputs can be considered in their temporal dimension, in order to access the driver's mental state variations. For example, the mental state can be affected in a way during and after the occurrence of a sudden event changing into fear/surprise, or in a different way during and after situations with high-density traffic (i.e., high spatio-temporal pressure and social pressure) leading to stress/anxiety.

The sensor outputs of the time after the driving context event maybe relevant for determining the comfort level of the driver. In this regard the disclosure differs from known systems at least in that it has a long-term function, which is not to avoid the current event but to assist the driver in the current event and especially in similar events in the future, in case a discomfort has been detected during (and in particular after) the current event.

It is further contemplated that the control device be equipped with machine learning techniques thereby using the current and historical data to determine or potentially predict (dis)comfort during driving.

The control device may further define a first fixed-length sliding window (SWL1) and a second fixed-length sliding window (SWL2) during which the first and second sensor outputs are recorded.

The control device may further define a first over-lap percentage (SWO1) and a second over-lap percentage (SWO2) that is different from the first over-lap percentage (SWO1).

The control device may sample the first and second sensor outputs with the first and second fixed-length sliding windows, respectively, in order to create the at least one reference data set.

The control device may determine the comfort level index value based on the product of the set of selected features within the first and second sliding windows, in particular based on the product of a first physiological feature and a second physiological feature, as:

$${CI}_{( SWL , SWO )} = X [ \begin{matrix}{x}_{1} \\ ⋮ \\ {x}_{n}\end{matrix} ] \cdot Y [ \begin{matrix}{y}_{1} \\ ⋮ \\ {y}_{n}\end{matrix} ] , \qquad ( 1 )$$

where xₙ the latest computed value of the first physiological feature X within the first sliding window and yₙ the latest computed value of the second physiological feature Y within the second sliding window.

The control device may determine a threshold (DT) for the comfort level index value based on the at least one reference data index, detect a discomfort state of the driver, when the comfort level index value exceeds the threshold (DT), and in particular, trigger a driver assistance system based on the detected discomfort state.

The control device may further define the mean value (M) and the standard deviation (SD) of the at least one reference index, in particular, over the first and/or second predetermined reference time period, and the threshold (DT) for the comfort level index value by the equation (2):

DT=M+SD (2),

The first sensor output may include a physiological measurement output, the physiological measurement output comprising a skin conductance level (SCL) signal of the driver, the second sensor output comprising an electrocardiographic (ECG) signal of the driver.

The control device may be configured to low-pass filter the skin conductance level (SCL) signal, in particular at 1 Hz or less, more particularly by using a zero time-lag second-order Butterworth filter, and/or normalize the skin conductance level (SCL) signal by dividing it by a predetermined reference skin conductance level (SCL).

The control device may determine the amplitude (AS) of the normalized SCL signal based on the difference between the maximal and minimal values of the normalized skin conductance level (SCL) signal within the first fixed-length sliding window.

The control device may determine the root mean square value (RMS) of the normalized SCL signal based on the following Equation (3):

$$RMS = \sqrt{\frac{1}{n} ( {x}_{1}^{2} + {x}_{2}^{2} + ⋯ + {x}_{n}^{2} )} , \qquad ( 3 )$$

where n is the number of normalized SCL values over the total duration of the first fixed-length sliding window and x represents the corresponding normalized SCL values.

The control device may further be configured to determine the heart rate (HR) of the driver based on the mean heart rate value of the ECG signal within the second fixed-length sliding window, and in particular, the heart rate variability (HRV) based on the beat-to-beat alterations in the heart rate.

The driving context detection unit of the control device may be configured to detect driving conditions including vehicle speed, vehicle acceleration, vehicle deceleration, lateral position, steering wheel reversal rate, and/or steering wheel angle.

The control device may further be configured to determine a first discomfort event if the duration of the activity is 30s or less, and determine a second discomfort event if the duration of the activity is at least 10 minutes.

The control device may, when the control device determines that a first discomfort event has occurred, determine the comfort level index value as a function of the first sensor and the first reference data set.

The control device may, when the control device determines that a second discomfort event has occurred, determine the comfort level index value as a function of the second sensor and the second reference data set.

The control device may record the at least one reference data set online.

The control device may, during a calibration of the threshold value (DT), acquire at least 10 mins of driving that is not determined to be a first discomfort event or a second discomfort event.

A system for a vehicle for determining a comfort level of a driver, the system comprising:

a control device according to any one of the preceding claims,
a first physiological sensor for measuring at least one first physiological feature of the driver, and
a second physiological sensor for measuring at least one second physiological feature of the driver.

The disclosure further relates to a system for a vehicle for determining a comfort level of a driver. The system comprises:
the first physiological sensor comprising: a conductance level (SCL) sensor, in particular with unpolarizable A_{g} or A_{g}Cₗ electrodes, more particularly with a surface of at least 30 mm²,
the second physiological sensor comprising an electrocardiographic (ECG) sensor, in particular with gold-plated active electrodes.

The disclosure further relates to a vehicle for determining a comfort level of a driver. The vehicle comprises:
a control device according to the control device discussed above or
a system according to the system discussed above.

The disclosure further relates to a method of determining a comfort level of a driver. The method comprises the steps of:
receiving sensor output of a first physiological sensor and a second physiological sensor, the first physiological sensor measuring at least one physiological feature of the driver and the second physiological sensor measuring at least one physiological feature of the driver,
creating at least one reference data set by recording the first sensor output over a first predetermined reference time period and recording the second sensor output over a second predetermined reference time period,
determining at least one reference index for the comfort level of the driver based on the at least one reference data set, and
determining a comfort level index value of the driver, the index value being determined as a function of the current first and/or second sensor output, and the at least one reference index.

The method may comprise further method steps where a reference skin conductance level (SCL) is determined before driving based on recording before starting driving the skin conductance level (SCL) signal of the driver at the driver position for a predetermined time period, in particular for at least 5 min.

It is intended that combinations of the above-described elements and those within the specification may be made, except where otherwise contradictory.

It is to be understood that both the foregoing general description and the following detailed description are exemplary and explanatory only and are not restrictive of the disclosure, as claimed.

The accompanying drawings, which are incorporated in and constitute a part of this specification, illustrate embodiments of the disclosure and together with the description, and serve to explain the principles thereof.

### BRIEF DESCRIPTION OF THE DRAWINGS

FIG. 1 shows a block diagram of a control device according to embodiments of the present disclosure;

FIG. 2 shows a schematic diagram of the data flow and data processing in the control device according to the embodiments of the present disclosure;

FIG. 3 shows a graph of the results of physiological features in terms of sensitivity and as a function of the driving event according to the embodiments of the present disclosure;

FIG. 4 shows a graph of the SCL as a function of time according to the embodiments of the present disclosure; and

FIG. 5 shows a graph of the HRV obtained before and after driving situations according to the embodiments of the present disclosure.

### DESCRIPTION OF THE EMBODIMENTS

Reference will now be made in detail to exemplary embodiments of the disclosure, examples of which are illustrated in the accompanying drawings. Wherever possible, the same reference numbers will be used throughout the drawings to refer to the same or like parts.

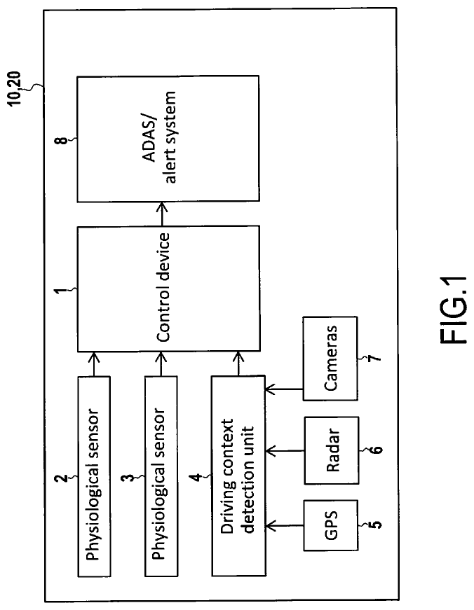

FIG. 1 shows a block diagram of a control device 1 according to embodiments of the present disclosure. The control device is part of a system 20. The system 20 is comprised by a vehicle 10.

The control device 1 may be connected to or comprise a data storage for storing a reference data set. The control device 1 may comprise an electronic circuit, a processor (shared, dedicated, or group), a combinational logic circuit, a memory that executes one or more software programs, and/or other suitable components that provide the described functionality. The control device 1 may additionally carry out further functions in the vehicle 1. For example, the control device may also act as the general purpose ECU (electronic control unit) of the vehicle.

The control device 1 receives data output of two or several physiological sensors 2, 3. Each of the physiological sensors 2, 3 measure at least one physiological feature of the driver. For example, the physiological sensor 2 measures the skin conductance level (SCL) and sensor 3 measures the electrocardiographic signal (ECG). Both may be recorded at 2000 Hz sampling rate for example. The SCL may be recorded with 30 mm² unpolarizable Ag/AgCl electrodes (e.g., from Clark Electromedical Instruments). It may be desirable to place the SCL sensors at least at the level of the second phalanx of the index and the third digit of the non-dominant hand of the driver. For ECG recording, gold-plated active electrodes may be used.

The control device 1 may low-pass filter the skin conductance level (SCL) signal, in particular at 1 Hz or less, for example by using a zero time-lag second-order Butterworth filter. The skin conductance level (SCL) signal may be normalized by dividing it by a predetermined reference skin conductance level (SCL). The control device 1 may also determine the amplitude (AS) of the normalized SCL signal based on the difference between the maximal and minimal values of the normalized skin conductance level (SCL) signal within a fixed-length sliding window or a time window having a fixed length and moves over the real time monitored electrophysical data. The SCL may be first recorded at rest during 5 min before driving, while the driver sits at the driving wheel without any stimulation and then averaged to be considered the reference. The control device 1 may also determine the root mean square value (RMS) of the normalized SCL signal based on the following Equation (3):

$$RMS = \sqrt{\frac{1}{n} ( {x}_{1}^{2} + {x}_{2}^{2} + ⋯ + {x}_{n}^{2} )} , \qquad ( 3 )$$

where n is the number of normalized SCL values over the total duration of the fixed-length sliding window and x represents the corresponding normalized SCL values. The heart rate (HR) of the driver may be determined based on the mean heart rate value of the ECG signal within a different and distinct fixed-length sliding window. In particular, the heart rate variability (HRV) may be determined based on the beat-to-beat alterations in the heart rate.

Furthermore, the control device receives driving context information from a driving context detection unit 4. The driving context information describes the current driving situation. Such driving situations may include, e.g., a situation in which the vehicle comprising the control device 1 overtakes another vehicle, a situation in which the vehicle comprising the control device 1 is approaching a traffic light, a situation in which the vehicle comprising the control device 1 approaches a pedestrian or a pedestrian approaches the vehicle, a situation in which the vehicle comprising the control device 1 is driven in the dark, a situation in which the vehicle comprising the control device 1 makes a left-turn (in right-hand traffic), or other driving situations which may challenge the comfort level of the driver.

In order to determine the driving context information, the driving context detection unit 4 receives information from a plurality of measurement units which monitor the environment of the vehicle, e.g. a GPS 5, a radar 6, one or several cameras 7, etc. The driving context detection unit 4 may be an electronic control device, e.g., a processor. It may also be provided by the general purpose ECU (electronic control unit) of the vehicle. It is further possible that the control device 1 comprises the driving context detection unit 4.

The control device 1 determines a driving context event based on the driving context information. In the examples mentioned above, such driving context events may be overtaking another vehicle, approaching a traffic light, approaching a pedestrian, driving in the dark, or making a left-turn (in right-hand traffic) among others.

As described in the following, the control device 1 determines a comfort level index value of the driver, in particular it detects a discomfort state of the driver, when the comfort level index value exceeds a predetermined threshold DT. In such a case, it may trigger an advanced driver assistance system (ADAS) 8. Generally the ADAS may assist driving based on the received comfort level index value. For example the ADAS may increasingly assist driving, i.e., increasingly overtake driving control tasks, in case the comfort level index value decreases, i.e., the driver's discomfort increases.

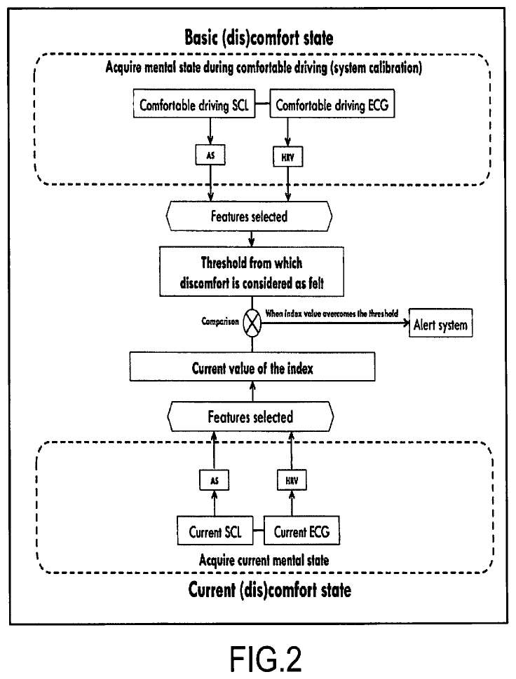

FIG. 2 shows a schematic diagram of the data flow and data processing in the control device according to embodiments of the present disclosure. The control device 1 determines a comfort level index value of the driver. Said value represents the current (dis)comfort state.

The comfort level index value is calculated based on the current sensor outputs, for example, a first and a second sensor, and may be based on a set of features including the amplitude (AS) of the normalized SCL, the root mean square value (RMS) of the normalized SCL signal, the heart rate (HR) of the driver, and/or the heart rate variability (HRV).

The electrophysiological features (heart rate variability and amplitude of normalized skin conductance level (SCL)) required for the index computation are computed within separate fixed-length sliding-windows moving over the real time monitored electrophysiological data. For each feature, some parameters have to be specified:
The sliding-window fixed-length (SWL).
The sliding windows over-lap percentages (SWO).
   The window length determines the number of data points per signal to be considered for a single window. The overlap factor determines the time offset between the first data points of two successive windows. While window length influences how much historical information is contained in a single window, the overlap factor influences how much historical information is shared among successive windows.

Sliding windows of 10 seconds with over-lap percentages of 99% moving over the SCL signal are used for the computation of the amplitude of normalized SCL. First the SCL may be low-pass filtered at 1 Hz using a zero time-lag second-order Butterworth filter for example to remove high frequency noise. Then, the filtered SCL signal may be divided by the reference to obtain the normalized SCL signal. The SCL may be first recorded at rest during 5 mins before driving, while the driver sits at the driving wheel without any stimulation and then averaged to be considered the reference. AS may correspond to the difference between the maximal and minimal values of the normalized SCL signal within the sliding window.

Sliding windows of 600 seconds (10 minutes) with over-lap percentages of 50% moving over the ECG signal may be used for the computation of the heart rate variability (HRV). HRV is computed as the standard deviation of the RR intervals over the total duration of the sliding window. This feature refers to the beat-to-beat alterations in heart rate that is widely used as an important marker of emotion regulatory ability.

Skin conductance level (SCL) and electrocardiographic signal (ECG), may both be recorded at 2000 Hz for example. SCL may be recorded with 30 mm2 unpolarizable Ag/AgCl electrodes (e.g., using devices from Clark Electromedical Instruments). The SCL sensors may be located at least at the level of the second phalanx of the index and the third digit of the non-dominant hand. For ECG recording, gold-plated active electrodes may be used.

At each time (each iteration), the index of (dis)comfort can be directly obtained from the product (or multiplication) of the two electrophysiological features. The latest value of each feature may be considered at each time.

The selection of the features used for the index value computation desirably depends on the type of driving situation (e.g., short term discomfort events where AS is used and/or long term discomfort events where HRV is used).

With continued reference to FIG. 2, the control device 1 further creates at least one reference data set by recording the multiple sensor outputs and the driving context information over a predetermined reference time period, in particular by recording the formerly determined comfort level index value of the driver. However, as mentioned above, the predetermined reference time period is different and distinct for each sensor and, further, may be dependent on the type of sensor used. For example, if a SCL sensor is used, an appropriate predetermined reference time period may be a sliding window of 10 seconds.

Based on the at least one reference data set, the control device 1 determines a reference index for the comfort level of the driver. The reference index represents the basic (dis)comfort state.

The predetermined reference time period may also comprise a plurality of driving sessions, e.g., the operating time of the vehicle during several weeks. Accordingly, a long-term monitoring of the driver can be achieved that is suitable to anticipate any negative long-term impact of the experienced (dis)comfort. Hence, the control device may take into account at least the accumulated operating time of the vehicle within one month as a reference time period. In other words, the comfort level index value of the driver can be correlated against historical data (e.g., collected in the last 1 month) represented by the reference index.

The reference index may be used to calculate a threshold for the comfort level index value. It is also possible that the threshold is calculated based on a predetermined selection of the set of features. The mean value (M) and the standard deviation (SD) of the index time series can be computed over a predetermined reference time period. Then, discomfort threshold (DT) will be computed following Equation (2):

DT=M+SD (2),

Accordingly, the index value is determined as a function of the current sensor outputs and the reference index, i.e., by correlating the current measurements against historical measurements. In this way it is possible to calibrate the currently determined (dis)comfort level. In particular the uncorrelated index value can be compared to the discomfort threshold (DT).

In addition, the index value is determined also as a function of the current driving context information. In this way it is possible to compare the currently determined (dis)comfort level only with those historical data which have been recorded in a corresponding driving event. Moreover, it is possible to determine the length of the current driving event such that the determination of the comfort level index value can be based on sensor outputs of the time before, during and after the event.

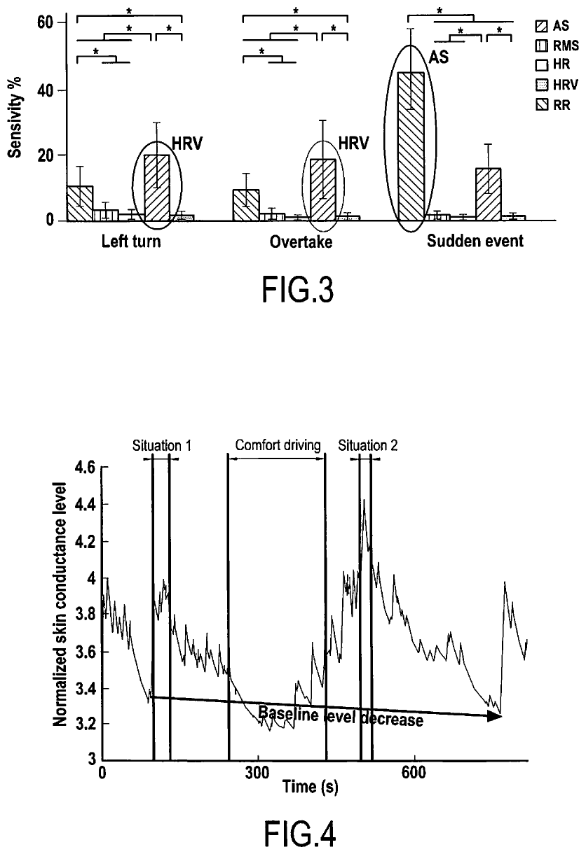

Turning to FIG. 3, a group of forty test drivers were subjected to a driving simulator experiment which included three driving scenarios: a safety-driving scenario used to compute a threshold beyond which discomfort in driving is considered, and two discomfort driving sessions. In the discomfort driving sessions, the test drivers were systematically exposed to different driving situations producing discomfort (truck overtaking, left-turn, sudden event).

The driving situations were repeated twice by varying the level of discomfort intensity (high level and high level plus one). For each level of intensity of each driving situation, 40 measures of the behavioural and physiological parameters investigated were obtained. In addition, after the driver's exposure to the driving situations, the feeling of subjective comfort/discomfort was monitored.

The results from the experiment are shown in FIG. 3. The results suggested that it could be more desirable to use the amplitude of the SCL (AS) signal for the computation of the (dis)comfort index for short terms and unexpected driving events such as the sudden insertion/braking of a car or the sudden crossing of a pedestrian. For longer situations, such as overtakings or left-turns under high density traffic conditions, it could be more pertinent to use the heart rate variability (HRV) physiological feature. Indeed, despite the fact that both the heart rate variability and the amplitude of the SCL signal provide us useful information about the level of stress and/or mental workload, the accuracy and sensitivity to the feeling of discomfort of these features differ as a function of the time period considered. It may also be desirable to combine heart rate variability and the amplitude of the SCL.

A complementary analysis was performed while using the data recorded during the high level plus one overtaking situations. For this high level plus one situation, the overtaking was repeated twice in order to produce a cumulative effect of discomfort. The results statistically confirmed that a residual effect of discomfort is observed for the skin conductance level. Indeed, the skin conductance level did not return to its baseline level during the very short time period (around 15-20 seconds) between the two consecutive trucks. However, such a residual effect of discomfort was not observed for heart rate variability (HRV).

FIG. 4 shows the results from a second complementary analysis that was performed over a longer time period. This time, the baseline physiological measures (skin conductance level and HRV) obtained during the very beginning and the very ending of the driving scenarios were compared. The mean skin conductance level was found to significantly decrease over the total duration of the scenario (around 10 min). Thus, a global long-term decrease in skin conductance level can be observed, whereas discomfort can be assessed through short-term changes. This last observation confirms the interest of using the amplitude of skin conductance within short temporal windows, especially for sudden event (duration shorter than 30 seconds).

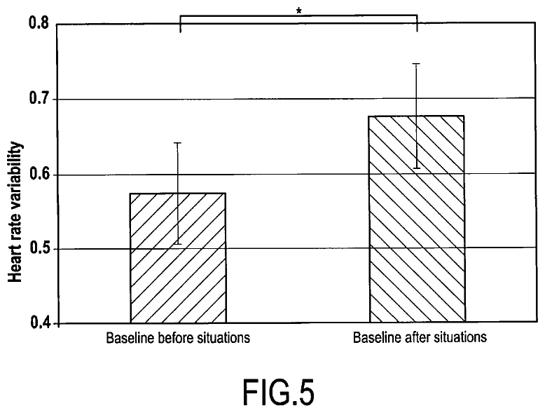

As shown in FIG. 5, the results also demonstrated that HRV significantly increased during the total duration of the scenario (around 10 min). This result brought the statistical evidence of the interest to use HRV when the objective is to detect the discomfort within a long period of time or to assess the cumulative effect of a series of uncomfortable situations.

Throughout the description, including the claims, the term “comprising a” should be understood as being synonymous with “comprising at least one” unless otherwise stated. In addition, any range set forth in the description, including the claims should be understood as including its end value(s) unless otherwise stated. Specific values for described elements should be understood to be within accepted manufacturing or industry tolerances known to one of skill in the art, and any use of the terms “substantially” and/or “approximately” and/or “generally” should be understood to mean falling within such accepted tolerances.

Although the present disclosure herein has been described with reference to particular embodiments, it is to be understood that these embodiments are merely illustrative of the principles and applications of the present disclosure.

It is intended that the specification and examples be considered as exemplary only, with a true scope of the disclosure being indicated by the following claims.

1. A control device for a vehicle for determining a comfort level of a driver,
the control device being configured to:
receive a first sensor output of a first physiological sensor measuring at least one first physiological feature of the driver, and
receive a second sensor output of a second physiological sensor measuring at least one second physiological feature of the driver,
create at least one reference data set by recording the first sensor output over a first predetermined reference time period and recording the second sensor output over a second predetermined reference time period being different than the first predetermined reference time period,
determine at least one reference index for the comfort level of the driver based on the at least one reference data set, and
determine a comfort level index value of the driver, the index value being determined as a function of the at least one reference index and the current first and/or second sensor output.

2. The control device according to claim 1 configured to:
receive driving context information from a driving context detection unit,
determine at least one reference index for the comfort level of the driver based on the at least one reference data set and, based on the current driving context information, determine the comfort level index value as a function of a first reference index and the current first sensor output or a second reference index and the current second sensor output, the first reference index being based on the first sensor output recorded over the first predetermined reference time period and the second reference index being based on the second sensor output recorded over the second predetermined reference time.

3. The control device according to claim 1 configured to:
define a first fixed-length sliding window and a second fixed-length sliding window during which the first and second sensor outputs are recorded.

4. The control device according to claim 3 configured to:
define a first over-lap percentage (SWO1) and a second over-lap percentage (SWO2) that is different from the first over-lap percentage (SWO1).

5. The control device according to claim 1 configured to:
sample the first and second sensor outputs with the first and second fixed-length sliding windows, respectively, in order to create the at least one reference data set.

6. The control device according to claim 1 configured to:
determine the comfort level index value based on the product of the set of selected features within the first and second sliding windows, in particular based on the product of a first physiological feature and a second physiological feature, as:
$${CI}_{( SWL , SWO )} = X [ \begin{matrix}{x}_{1} \\ ⋮ \\ {x}_{n}\end{matrix} ] \cdot Y [ \begin{matrix}{y}_{1} \\ ⋮ \\ {y}_{n}\end{matrix} ] , \qquad ( 1 )$$
where xₙ the latest computed value of the first physiological feature X within the first sliding window and yₙ the latest computed value of the second physiological feature Y within the second sliding window.

7. The control device according to claim 1 configured to:
determine a threshold for the comfort level index value based on the at least one reference data index,
detect a discomfort state of the driver when the comfort level index value exceeds the threshold, and in particular, trigger a driver assistance system based on the detected discomfort state.

8. The control device according to claim 1 configured to determine:
a mean value (M) and a standard deviation (SD) of the at least one reference index, in particular, over the first and/or second predetermined reference time period, and the threshold for the comfort level index value by the equation:
DT=M+SD.

9. The control device according to claim 1, wherein the first sensor output includes a physiological measurement output, the physiological measurement output comprising a skin conductance level signal of the driver, the second sensor output comprising an electrocardiographic signal of the driver.

10. The control device according to claim 1 configured to:
low-pass filter the skin conductance level signal, in particular at 1 Hz or less, more particularly by using a zero time-lag second-order Butterworth filter, and/or normalize the skin conductance level signal by dividing it by a predetermined reference skin conductance level.

11. The control device according to claim 3 configured to:
determine the amplitude of the normalized SCL signal based on the difference between the maximal and minimal values of the normalized skin conductance level signal within the first fixed-length sliding window.

12. The control device according to claim 3 configured to:
determine the root mean square value of the normalized SCL signal based on the following Equation (3):
$$RMS = \sqrt{\frac{1}{n} ( {x}_{1}^{2} + {x}_{2}^{2} + ⋯ + {x}_{n}^{2} )} , \qquad ( 3 )$$
where n is the number of normalized SCL values over the total duration of the first fixed-length sliding window and x represents the corresponding normalized SCL values.

13. The control device according to claim 3 wherein the first sensor output includes a physiological measurement output, the physiological measurement output comprising a skin conductance level signal of the driver, the second sensor output comprising an electrocardiographic signal of the driver, the control device being configured to determine:
a heart rate of the driver based on a mean heart rate value of the ECG signal within the second fixed-length sliding window, and in particular
a heart rate variability based on the beat-to-beat alterations in the heart rate.

14. The control device according to claim 2, wherein the driving context detection unit is configured to detect driving conditions including vehicle speed, vehicle acceleration, vehicle deceleration, lateral position, steering wheel reversal rate, and/or steering wheel angle.

15. The control device according to claim 14 configured to determine a first discomfort event if the duration of the activity is 30s or less, and determine a second discomfort event if the duration of the activity is at least 10 minutes.

16. The control device according to claim 15, wherein when the control device determines that a first discomfort event has occurred, the comfort level index value is determined as a function of the first sensor output and the first reference data set.

17. The control device according to claim 15, wherein when the control device determines that a second discomfort event has occurred, the comfort level index value is determined as a function of the second sensor output and the second reference data set.

18. The control device according to claim 1, wherein the at least one reference data set recording is online.

19. The control device according to claim 15, wherein during a calibration of the threshold value, the control device acquires at least 10 mins of driving that is not determined to be a first discomfort event or a second discomfort event.

20. A system for a vehicle for determining a comfort level of a driver, the system comprising:
a control device according to claim 1,
a first physiological sensor for measuring at least one first physiological feature of the driver, and
a second physiological sensor for measuring at least one second physiological feature of the driver.

21. The system according to claim 10, the first physiological sensor comprising: a conductance level sensor, in particular with unpolarizable A_{g} or A_{g}Cₗ electrodes, more particularly with a surface of at least 30 mm², the second physiological sensor comprising an electrocardiographic sensor, in particular with gold-plated active electrodes.

22. A vehicle comprising:
a control device according to claim 1.

23. A method of determining a comfort level of a driver, the method comprising the steps of:
receiving sensor output of a first physiological sensor and a second physiological sensor, the first physiological sensor measuring at least one physiological feature of the driver and the second physiological sensor measuring at least one physiological feature of the driver,
creating at least one reference data set by recording the first sensor output over a first predetermined reference time period and recoding the second sensor output over a second predetermined reference time period,
determining at least one reference index for the comfort level of the driver based on the at least one reference data set, and
determining a comfort level index value of the driver, the index value being determined as a function of the current first and/or second sensor output, and the at least one reference index.

24. The method according to claim 23, wherein
a reference skin conductance level is determined before driving based on recording before starting driving the skin conductance level signal of the driver at the driver position for a predetermined time period, in particular for at least 5 min.
